(19) United States
(12) Patent Application Publication
(10) Pub. No.: US 2025/0065311 A1
(43) Pub. Date: Feb. 27, 2025

(54) SULFIDATED NOBLE METAL ON METAL DOPED SUPPORT AS A HYDROGENATION AND DEHYDROGENATION CATALYST

(71) Applicant: Chevron U.S.A. Inc., San Ramon, CA (US)

(72) Inventors: Trenton J. Otto, El Sobrante, CA (US); Bi-Zeng Zhan, Albany, CA (US); Steven Xuqi Song, El Cerrito, CA (US); Christopher Declan Lane, Kensington, CA (US)

(21) Appl. No.: 18/454,048

(22) Filed: Aug. 22, 2023

(51) Int. Cl. B01J 27/045; B01J 23/10; C01B 3/00; C07C 5/10

(52) U.S. Cl. CPC B01J 27/045; B01J 23/10; C01B 3/0015; C07C 5/10; C07C 2521/04; C07C 2523/10; C07C 2527/045

(57) ABSTRACT

This disclosure pertains to a supported noble metal catalyst containing noble metal component and a sulfur-containing component being supported on a non-noble metal doped inorganic oxide carrier and uses thereof. The catalyst may be used for the hydrogenation of an aromatic compound. The present disclosure further relates to a process for the partial or complete dehydrogenation of perhydrogenated or partially hydrogenated cyclic hydrocarbons to produce hydrogen.

### FIELD

This disclosure relates to a supported noble metal- and sulfur-containing catalyst having a non-noble metal-doped support. The catalyst may be used for hydrogenation or dehydrogenation reactions. The catalyst may be used for aromatics saturation, base oil hydrofinishing, catalytic reforming, hydrodenitrogenation, hydrodeoxygenation, naphtha dehydrogenation for chemicals, hydrogenation/dehydrogenation of high aromatics-containing refinery streams such as light cycle oil (LCO), or partial or complete dehydrogenation of perhydrogenated or partly hydrogenated cyclic hydrocarbons to produce hydrogen. The present disclosure also relates to a process for partial or complete dehydrogenation of perhydrogenated or partly hydrogenated cyclic hydrocarbons to produce hydrogen.

### BACKGROUND

Hydrogen is one of the more important options for future clean energy. Unfortunately, storage and transportation of hydrogen fuel from its production location to, for example, a hydrogen gas station or other storage facility are often inefficient and/or costly today. This issue is well recognized as one key barrier for hydrogen to be deployed at a large scale. The storage of hydrogen in liquid organic hydrogen carrier (LOHC) systems has numerous advantages over conventional storage systems (e.g., compression and liquefaction technologies). Most importantly, hydrogen storage and transport in the form of LOHC system enables the use of the existing infrastructure for fuels. From a thermodynamic point of view, hydrogen storage in LOHC system requires an exothermic hydrogenation step and an endothermic dehydrogenation step (i.e., one which requires an input of heat, at a temperature where the dehydrogenation of the carrier can proceed with adequate reaction rates).

LOHC systems are generally based on a pair of aromatic and alicyclic compounds or a pair of heteroaromatic and heterocyclic compounds, where reversible hydrogen storage and release are achieved by catalytic hydrogenation of hydrogen-lean molecule (LOHC−) and catalytic dehydrogenation of the corresponding full hydrogenation product (LOHC+), respectively. Representative LOHC compound pairs include benzene and cyclohexane, toluene and methylcyclohexane, naphthalene and decalin, N-ethyl carbazole (H₀-NEC) and dodecahydro-N-ethyl carbazole (H₁₂-NEC), benzyltoluene (H₀-BT) and perhydro-benzyltoluene (H₁₂-BT), and dibenzyltoluene (H₀-DBT) and perhydro-dibenzyltoluene (H₁₈-DBT).

For hydrogenation and dehydrogenation, supported noble metal catalysts, especially platinum (Pt), palladium (Pd), and ruthenium (Ru) are often used. Despite continued advancements in the selection and preparation of hydrogenation/dehydrogenation catalysts, known dehydrogenation catalysts suffer from efficiency and stability issues. These issues are typically exacerbated during the prolonged dehydrogenation reactions of LOHC technologies.

Therefore, developing a suitable catalyst for LOHC systems using a single heterogeneous catalyst for both dehydrogenation and hydrogenation under mild conditions is highly desirable.

### SUMMARY

In a first aspect, the present disclosure relates to a supported noble metal catalyst comprising: (a) a carrier, wherein the carrier is a shaped structure defined by a shape and comprises an inorganic oxide and a non-noble metal dopant, wherein the non-noble metal dopant is present in an amount of from 0.01 to 10 wt. % of catalyst, measured as oxide; (b) a noble metal component, wherein a content of the noble metal component is from 0.01 to 2 wt. %, measured as elemental metal; and (c) a sulfur-containing component, wherein a content of the sulfur-containing component is from 0.01 to 2 wt. %, measured as elemental sulfur.

In a second aspect, the present disclosure relates to a process for the hydrogenation of an aromatic compound comprising (a) preparing a mixture of an aromatic compound and a supported noble metal catalyst according to the first aspect; and (b) contacting the mixture obtained in (a) with hydrogen at a temperature comprised in the range of from 100° C. to 400° C.

In a third aspect, the present disclosure relates to a process for producing hydrogen, the process comprising: contacting a feed comprising a perhydrogenated or partly hydrogenated cyclic hydrocarbon under dehydrogenation conditions with a supported noble metal catalyst according to the first aspect.

### BRIEF DESCRIPTION OF THE DRAWINGS

FIG. 1 is a plot of degree of dehydrogenation (DoDH) of perhydro-dibenzyltoluene vs. time on stream at 650° F. for various catalysts.

### DETAILED DESCRIPTION

### Definitions

As used herein, and unless the context dictates otherwise, the following terms have the meanings as specified below.

The term “catalyst” means a substance that alters the rate of a chemical reaction. A catalyst may either increase the chemical reaction rate (i.e., a “positive catalyst”) or decrease the reaction rate (i.e., a “negative catalyst”). Catalysts participate in a reaction in a cyclic fashion such that the catalyst is cyclically regenerated. “Catalytic” means having the properties of a catalyst.

The term “dopant” or “doping agent” or “doping element” is chemical compound which is added to or incorporated within a catalyst base material to optimize catalytic performance (e.g., increase or decrease catalytic activity). As compared to the undoped catalyst, a doped catalyst may increase or decrease the selectivity, conversion, and/or yield of a reaction catalyzed by the catalyst. Dopants which increase catalytic activity are referred to as “promoters” while dopants which decrease catalytic activity are referred to as “poisons”. The non-noble metal dopant can be a rare earth element, a transition metal, other metal, or any combination thereof. The dopant may be present in the catalyst in any form and may be derived from any suitable source of the element (e.g., chlorides, bromides, iodides, nitrates, oxynitrates, oxyhalides, acetates, formates, hydroxides, carbonates, phosphates, sulfates, alkoxides, and the like.)

The term “carrier” or “support” interchangeably refers to conventional materials that are typically a solid with a high surface area, to which catalyst materials are affixed. Support materials may be inert or participate in the catalytic reactions and may be porous or non-porous.

The term “noble metal” refers to metals that are highly resistant to corrosion and/or oxidation. Group VIII noble metals include ruthenium (Ru), osmium (Os), rhodium (Rh), iridium (Ir), palladium (Pd), and platinum (Pt).

The term “rare earth” refers to an element that is scandium (Sc), yttrium (Y), or a lanthanide element. The term “lanthanide element” refers to an element in the lanthanide series of the periodic table of elements. The lanthanide series can have an atomic number 57 (for lanthanum) to 71 (for lutetium). Elements included in this series are lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

As used herein, the term “impregnated” or “impregnation” refers to permeation of the catalytic material into the porous structure of the support material.

The terms “benzyltoluene” and “dibenzyltoluene” include isomers of the compounds mentioned. In addition, the terms benzyltoluene and dibenzyltoluene also include substituted benzyl- or dibenzyltoluenes in which one or both benzyl groups are substituted by one or more groups selected from alkyl groups, such as methyl or ethyl groups, aryl groups, such as phenyl groups, and heteroaryl groups, such as pyridinyl groups.

The term “light cycle oil” and its acronym “LCO” as used herein refers to a middle distillate produced by fluid catalytic cracking units. The nominal boiling range for this stream is, for example, in the range of about 215° C. to 350° C. (e.g., 220° C. to 350° C., 215° C. to 343° C., or 220° C. to 343° C., 215° C. to 330° C., or 220° C. to 330° C.). LCO is an aromatic rich stream and the aromatic content in LCO can vary from about 60 wt. % to 90 wt. % depending upon operational severity and type of feedstock.

The terms “wt. %”, “vol. %”, or “mol. %” refer to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

### Catalyst Composition

The supported noble metal catalyst composition includes (a) a carrier, wherein the carrier is a shaped structure defined by a shape and comprises an inorganic oxide and a non-noble metal dopant, (b) a noble metal component, and (c) a sulfur-containing component.

The carrier may be in a form selected from the group consisting of spheres, cylindrical pellets, and extrudates. In one embodiment, the support is in the form of extrudates.

The carrier component of the catalyst is prepared by any of the suitable methods known to those skilled in the art for preparing shaped porous catalyst supports used to carry catalytically active metals. The carrier or shaped inorganic oxide carrier comprises a porous refractory oxide or inorganic oxide component such as alumina (Al₂O₃), silica (SiO₂), titania (TiO₂), zirconia (ZrO₂), and physical mixtures or chemical combinations thereof. The preferred inorganic oxide for the shaped inorganic oxide carrier of the catalyst is one selected from the group consisting of alumina, silica, alumina-silica, and titania. Among these, the most preferred inorganic oxide is alumina. The alumina may be selected from alpha-alumina, beta-alumina, gamma-alumina, delta-alumina, theta-alumina, or any combination thereof. In some embodiments, the catalyst composition comprises an inorganic oxide selected from alumina, silica, titania, zirconia, and any mixture thereof, present in the catalyst composition in an amount within a range of from 90 wt. % to 99.9 wt. %, calculated as oxide on a calcined basis (e.g., 92 wt. % to 99 wt. %, or 95 wt. % to 99 wt. %).

The inorganic oxide of the carrier is formed into a shaped structure by any known suitable method. The extruded shaped structures typically are prepared by mixing an inorganic oxide powder with water and one or more additives to form a mixture having plastic properties and forming the mixture into extrudates by any of the known extrusion methods. The formed extrudates can be cylinders, lobed-shaped, and twisted shapes having nominal extrudate diameters in the range of from 0.5 mm to 25 mm and extrudate lengths in the range of from 1 mm to 50 mm. The average aspect ratio is usually at most 10, typically at most 8, for instance at most 5. The average length to diameter aspect ratio of the extrudates may range from 1:1 to 20:1 (e.g., 2:1 to 9:1, or 4:1 to 8:1).

Spherically shaped structures of the inorganic oxide carrier can be made by the application of any of the known granulation methods, which use an inclined rotating disk or pan that is fed particles of the inorganic oxide while spraying a cohesive slurry onto the particles. By this method the particles are formed into spherically shaped particles. The spherically shaped carrier particles can have diameters in the range of from 0.5 mm to 25 mm.

Dry tableting methods may also be used to prepare the shaped carrier structure. In this method, cylindrical pellets or pills of the inorganic oxide are made by pressing a dry inorganic oxide powder that is optionally mixed with additives such as a lubricant and a binder, between two punches in a tableting press. Carrier particles that are cylindrical pellets can have pellet diameters in the range of from 0.5 mm to 25 mm and pellet lengths in the range of from 1 mm to 50 mm.

The shaped support particles are then dried under standard drying conditions that can include a drying temperature in the range of from 50° C. to 200° C. (e.g., 75° C. to 175° C., or 90° C. to 150° C.).

After drying, the shaped support particle is calcined under standard calcination conditions that include a calcination temperature in the range of from 200° C. to 800° C. (e.g., 300° C. to 700° C., or 400° C. to 600° C.).

The calcined shaped support is preferably porous and has a high surface area. The calcined shaped support can have Brunauer-Emmett-Teller (“BET”) surface area of at least 100 m²/g (e.g., 100 m²/g to 350 m²/g, or 125 m²/g to 250 m²/g). The BET surface area may be determined by a method in accordance with ASTM D3663. The pore volume of the calcined shaped support can be in a range of from 0.2 cm³/g to 2.0 cm³/g (e.g., 0.4 cm³/g to 1.5 cm³/g, or 0.4 cm³/g to 1.2 cm³/g). Pore volumes disclosed herein may be measured by two methods, namely, the mercury method (ASTM 4284) and the nitrogen method (ASTM D4222).

The carrier is doped with a non-noble metal dopant. The non-noble metal dopant can be an element selected from the group consisting of titanium (Ti), zirconium (Zr), chromium (Cr), manganese (Mn), iron (Fe), cobalt (Co), nickel (Ni), copper (Cu), zinc (Zn), tin (Sn), rare earth metals, and any combination thereof. A rare earth dopant may be an element selected from the group consisting of scandium (Sc), yttrium (Y), lanthanum (La), praseodymium (Pr), neodymium (Nd), samarium (Sm), gadolinium (Gd), ytterbium (Yb), or metal oxides thereof. In some embodiments, the dopant comprises lanthanum (e.g., consists essentially of lanthanum or consists of lanthanum).

The non-noble metal dopant is typically present in an amount of from 0.01 to 10 wt. % (e.g., 0.1 to 10 wt. %, or 0.1 to 8 wt. %, or 1 to 10 wt. %, or 1.5 to 8 wt. %, or 2 to 6 wt. %), measured as the oxide. The foregoing amounts are based on the total weight of the catalyst composition.

Preferably, the carrier inherently is substantially free of sulfur-containing compounds prior to impregnation with the sulfur-containing compound. As used herein, the term “substantially free” means that there is generally less than 0.5 wt. %, including less than about 0.25 wt. %, or less than about 0.1 wt. %, of the referenced component. The term “substantially free of sulfur-containing compounds” includes “free of sulfur-containing compounds”.

The noble metal component comprises any noble metal-containing compound, complex, or the like which, upon calcination or use of the catalyst decomposes or otherwise converts to a catalytically active form, usually the metal or the metal oxide. In some embodiments, the noble metal can be ruthenium (Ru), rhodium (Rh), iridium (Ir), palladium (Pd), platinum (Pt), or any combination thereof. In other embodiments, the noble metal can be ruthenium, iridium, palladium, platinum, or any combination thereof. In further embodiments, the noble metal can be platinum. The concentration of the noble metal component can vary but will typically be from 0.01 wt. % to 2 wt. % (e.g., 0.1 to 2 wt. %, or 0.1 to 1.0 wt. %, or 0.3 to 0.6 wt. %), calculated as elemental metal.

The sulfur-containing component comprises any sulfur-containing compound, complex, or the like. The concentration of the sulfur-containing component can vary but will typically be from 0.01 wt. % to 2.0 wt. % (e.g., 0.1 to 1.0 wt. %, or 0.3 to 0.6 wt. %), calculated as elemental sulfur. In some embodiments, the sulfur-containing component includes sulfates, disulfates, sulfides, persulfates, and combinations thereof. Without wishing to be bound by theory, it is believed that the sulfur-containing component imparts resistance against deactivation to the noble metal component during catalytic reactions.

The noble metal component and sulfur-containing component may be present in a molar ratio of from 1:10 to 10:1 (e.g., 1:1 to 1:5), and wherein the sulfur-containing component is calculated as elemental sulfur.

### Method of Making the Catalyst Composition

Formation of a doped support will normally involve at least co-precipitating, co-kneading, and/or mixing of an inorganic oxide source and non-noble metal dopant source to form an extrudable paste. If required additional heat is introduced in the process to remove additional water. The mixture is extruded in the form of spheres or extrudates, dried, and calcined (in the presence or absence of steam).

A dopant (e.g., La₂O₃, TiO₂, ZrO₂, ZnO, SnO₂) can be doped into support materials either by an incipient wetness impregnation method or a co-precipitation method. Without wishing to be bound by theory, it is noted that dopants loaded onto support materials by impregnation methods are mainly present on the surface of the support, whereas dopants loaded onto support materials by co-precipitation methods are more homogeneously distributed in the support matrix. Dopant precursors can be used in the doping processes. For example, the dopant precursors can be nitrates, acetates, chlorides, oxalates, etc. After dopant impregnation (in which the solution containing dopant precursors is impregnated onto the support) or co-precipitation (in which the solution containing dopant precursors and support precursor is precipitated out using a precipitator such as ammonia, urea, or other alkaline agents), the resulting support materials can be calcined at about 400° C. to 600° C. for about 1-5 hours.

The noble metal component and the sulfur-containing component can be introduced onto the extrudates in one or more steps.

Preparation of a bi-component (i.e., noble metal and sulfur-containing components) impregnated support typically comprises impregnating the support material with a noble metal component precursor and a sulfur-containing component precursor in solution, wherein both precursors are either in the same solution or separate solutions. The noble metal component and the sulfur-containing component can be impregnated at the same time or separately using an incipient wetness technique.

Incipient wetness impregnation techniques, also called capillary impregnation or dry impregnation, are commonly used for the synthesis of heterogeneous materials. Typically, a metal precursor is dissolved in an aqueous or organic solution and then the metal-containing solution is added to a catalyst support, containing the same pore volume as the volume of the solution that was added. Capillary action draws the solution into the pores of the support. Solution added in excess of the support pore volume causes the solution transport to change from a capillary action process to a diffusion process, which is much slower. The catalyst can then be dried and calcined to drive off the volatile components within the solution, depositing the metal on the catalyst surface.

In the preparation of aqueous solutions of the noble metal component precursors, noble metal component salts of the noble metal component are used, such as their corresponding nitrate, amine complex (e.g., tetraammine), halide (e.g., chloride), carboxylate (e.g., acetate), or combinations thereof.

In the preparation of aqueous solutions of the sulfur-containing component, a sulfur-containing precursor is used such as sulfuric acid, ammonium sulfate, ammonium sulfide, ammonium persulfate, or combinations thereof. The sulfur-containing precursor is preferably sulfuric acid, ammonium sulfate, or a combination thereof. In some embodiments, the measured aqueous solubility of sulfur-containing component precursors is at least 50 g/100 mL of water.

In some embodiments, the preparation of an impregnated shaped support body requires an aqueous mixture containing the noble metal component precursor, the sulfur-containing component precursor, and the shaped support body. In such embodiments, both precursors are in contact with the shaped support body at the same time.

In further embodiments, an aqueous solution containing the noble metal component precursor and the sulfur-containing component precursor is prepared in advance (such as by pre-mixing both components) prior to contact with the shaped support body for impregnation.

In other embodiments, the noble metal component and the sulfur-containing component are impregnated separately onto the same shaped support body. For example, in one embodiment, the sulfur-containing component is first impregnated onto the shaped support body to form a sulfur-containing component impregnated shaped support body. This support is further modified upon exposure to a solution of a noble metal component precursor to allow additional impregnation of the noble metal component onto the already impregnated shaped support body to generate the bi-component impregnated inorganic oxide material described herein.

In another embodiment, the sulfur-containing component added to the base material during the extrusion phase of the catalyst preparation.

Following treatment of the support with the noble metal component precursor solution and sulfur-containing component precursor solution or solutions, the impregnated support is dried, such as by heat treating the support at elevated temperature (e.g., 100° C. to 150° C.) for a period of time (e.g., 1 to 3 hours), and then calcined to convert the noble metal component and sulfur-containing component to more catalytically active forms. An exemplary calcination process involves heat treatment in air at a temperature of from 400° C. to 550° C. for 1-3 hours. The above process can be repeated as needed to reach the desired level of noble metal component and sulfur-containing component impregnation.

### Use of the Catalyst Composition

The supported noble metal catalyst composition of the present disclosure may be used generally for all chemical reactions for which noble metal catalysts are suitable. Such reactions may include isomerization, oxidation, hydrogenolysis (e.g., hydrodesulfurization), hydrogenation/dehydrogenation, base oil hydrofinishing, hydrodewaxing, reforming, hydrodeoxygenation, and hydrodenitrogenation reactions.

In one embodiment, the supported noble metal catalyst of the present disclosure may be used for selective hydrogenation of an aromatic compound or an unsaturated compound.

The catalyst may be used in an amount of from 0.01 parts by weight to about 10 parts by weight based on 100 parts by weight of the aromatic compound or the unsaturated compound. If the amount of the catalyst is less than about 0.01 part by weight, the hydrogenation time may be increased and the reaction may not be completed, and if it is more than about 10 parts by weight, the hydrogenation may be accelerated, but the selectivity of the product may be lowered due to generation of by-products.

The aromatic compound or the unsaturated compound may include at least one selected from benzene, toluene, benzyltoluene, dibenzyltoluene, xylene, naphthalene, fluorene, and N-alkylcarbazoles (e.g., N-methylcarbazole, N-ethylcarbazole, N-propylcarbazole), light cycle oils, and partially saturated derivatives thereof. In an embodiment, the aromatic compound or the unsaturated compound may be toluene, dibenzyltoluene, and N-ethylcarbazole.

The hydrogenation reaction is generally carried out at a temperature of from 100° C. to 350° C. (e.g., 120° C. to 300° C., 150° C. to 250° C.). The pressure employed for the hydrogenation reaction is generally from 0.1 MPa to 10 MPa (e.g., 0.5 MPa to 8 MPa, or 1 MPa to 5 MPa).

The hydrogenation reaction can be conducted with or without a solvent. When a solvent is used in the hydrogenation, aliphatic compounds including n-pentane, n-hexane, n-heptane, n-octane, n-nonane, n-decane, etc. and cycloalkane hydrocarbons including cyclohexane, cycloheptane, cyclooctane, cyclononane, cyclodecane, etc. may be used. In an embodiment, cyclohexane may be used as the solvent for the hydrogenation. When using a solvent, the solvent can be from 20 wt. % to 95 wt. % (e.g., 30 wt. % to 95 wt. %, or 40 wt. % to 95 wt. %, or 50 wt. % to 95 wt. %, or 60 wt. % to 95 wt. %, or 70 wt. % to 95 wt. %) of the feed mixture.

The hydrogenation reaction may be carried out in a wide variety of batch, semi-batch, and continuous reactor systems. The configuration of the reactor is not critical. Suitable conventional reactor configurations include, for example, stirred tank reactors, fixed bed reactors, trickle bed reactors, fluidized bed reactors, bubble flow reactors, plug flow reactors, buss loop reactors, and parallel flow reactors.

The hydrogenation reaction can be partial or complete and preferably the hydrogenation reaction is complete in one or more hydrogenation/dehydrogenation cycles, that is to say that all of the double bonds capable of being hydrogenated present in an aromatics-containing liquid organic hydrogen carrier (LOHC) formulation are completely hydrogenated.

In one embodiment, the supported metal catalyst of the present disclosure may be used for partial or complete dehydrogenation of perhydrogenated or partially dehydrogenated cyclic hydrocarbons.

The cyclic hydrocarbons are in either perhydrogenated or partly hydrogenated form, meaning that they have only a small number of carbon-carbon multiple bonds, if any.

The perhydrogenated or partly hydrogenated cyclic hydrocarbons may, in addition to carbon and hydrogen, also contain heteroatoms, such as nitrogen. The perhydrogenated or partly hydrogenated cyclic hydrocarbons preferably do not contain any oxygen.

In a preferred embodiment, the perhydrogenated or partly hydrogenated cyclic hydrocarbon is selected from the group consisting of cyclohexane, methylcyclohexane, decalin, perhydrogenated or partly hydrogenated benzyltoluene and perhydrogenated or partly hydrogenated dibenzyltoluene and isomers thereof, perhydrogenated or partly hydrogenated light cycle oils. In addition, preferred embodiments are perhydrogenated or partly hydrogenated N-alkylcarbazoles, preferably perhydrogenated or partly hydrogenated N-ethylcarbazole, N-methylcarbazole and N-propylcarbazole, especially perhydrogenated or partly hydrogenated N-ethylcarbazoles.

Partly hydrogenated benzyltoluene includes benzyltoluene compounds in which at least one carbon-carbon double bond of the benzyltoluene has been replaced by a carbon-carbon single bond. Perhydrogenated benzyltoluene includes benzyltoluene compounds in which the carbon-carbon double bonds have been replaced by carbon-carbon single bonds. Examples of partly hydrogenated benzyltoluenes are 1-cyclohexylmethyl-2-methylbenzene, 1-cyclohexylmethyl-3-methylbenzene, 1-cyclohexylmethyl-4-methylbenzene, 1-benzyl-2-methylcyclohexane, 1-benzyl-3-methylcyclohexane, 1-benzyl-4-methylcyclohexane, 1-(1-cyclohexenylmethyl)-2-methylbenzene, 1-(1-cyclohexenylmethyl)-3-methylbenzene, 1-(1-cyclohexenylmethyl)-4-methylbenzene, 1-(1,3-cyclohexadienylmethyl)-2-methylbenzene, 1-(1,3-cyclohexadienylmethyl)-3-methylbenzene and 1-(1,3-cyclohexadienylmethyl)-4-methylbenzene.

Examples of perhydrogenated benzyltoluenes are 1-cyclohexylmethyl-2-methylcyclohexane, 1-cyclohexylmethyl-3-methylcyclohexane and 1-cyclohexylmethyl-4-methylcyclohexane.

A partly hydrogenated dibenzyltoluene includes any dibenzyltoluene compound in which at least one carbon-carbon double bond of the dibenzyltoluene has been replaced by a carbon-carbon single bond. A perhydrogenated dibenzyltoluene includes any dibenzyltoluene compound in which all carbon-carbon double bonds have been replaced by carbon-carbon single bonds. Examples of partly hydrogenated dibenzyltoluene are 1-benzyl-3-(cyclohexylmethyl)-5-methylbenzene, (5-methyl-1,3-phenylene)bis(methylene)dicyclohexane, 1-benzyl-4-(cyclohexylmethyl)-2-methylbenzene, (2-methyl-1,4-phenylene)bis(methylene)dicyclohexane, 2-benzyl-4-(cyclohexylmethyl)-1-methylbenzene, (4-methyl-1,3-phenylene)bis(methylene)dicyclohexane, 1-benzyl-3-(cyclohexylmethyl)-2-methylbenzene, (2-methyl-1,3-phenylene)bis(methylene)dicyclohexane, 1-benzyl-2-(cyclohexylmethyl)-4-methylbenzene, (4-methyl-1,2-phenylene)bis(methylene)dicyclohexane, 1-benzyl-3-(1-cyclohexenylmethyl)-5-methylbenzene and 1-benzyl-3-(1,3-cyclohexadienylmethyl)-5-methylbenzene. Examples of perhydrogenated dibenzyltoluene are (5-methylcyclohexane-1,3-diyl)bis(methylene)dicyclohexane, (2-methylcyclohexane-1,4-diyl)bis(methylene)dicyclohexane, (4-methylcyclohexane-1,3-diyl)bis(methylene)dicyclohexane, (2-methylcyclohexane-1,3-diyl)bis(methylene)dicyclohexane and (4-methylcyclohexane-1,2-diyl)bis(methylene)dicyclohexane.

A partly hydrogenated N-alkylcarbazole, such as N-ethylcarbazole, includes any N-alkylcarbazole in which at least one carbon-carbon double bond present therein has been replaced by a carbon-carbon single bond. A perhydrogenated N-alkylcarbazole, such as N-ethylcarbazole, includes any N-alkylcarbazole in which all carbon-carbon double bond present therein have been replaced by carbon-carbon single bonds. Examples of partly hydrogenated N-ethylcarbazole are 9-ethyl-2,3,4,9-tetrahydro-1H-carbazole, 9-ethyl-2,3,4,5,6,9-hexahydro-1H-carbazole, 9-ethyl-2,3,4,5,6,7,8,9-octahydro-1H-carbazole and 9-ethyl-2, 3, 4,4a,5,6,7,8,9,9a-decahydro-1H-carbazole. Perhydrogenated N-ethylcarbazole is, for example, 9-ethyldodecahydro-1H-carbazole.

The dehydrogenation may be partial or complete. The dehydrogenation is preferably complete. Dehydrogenation is complete when fully saturated carbocycyl or heterocyclyl radicals (e.g., cyclohexyl or piperidinyl radicals) or partially saturated carbocyclyl or heterocyclyl radicals (e.g., cyclohexenyl or dihydropyridyl radicals) are converted to the corresponding aromatic form (e.g., phenyl or pyridyl radicals).

In a preferred embodiment, the dehydrogenation is performed continuously in a reactor selected from a fixed-bed reactor, a flow-bed reactor, or a fluidized bed reactor, preferably a fixed-bed reactor.

The dehydrogenation is preferably carried out at a temperature of from 200° C. to 400° C. (e.g., 250° C. to 380° C., or 300° C. to 360° C.). The dehydrogenation is preferably performed at a pressure of from 50 kPa to 500 kPa (e.g., 100 kPa to 400 kPa). The liquid hourly space velocity is preferably in a range of from 0.1 h⁻¹ to 3 h⁻¹ (e.g., 1 h⁻¹ to 2 h⁻¹). For better removal of hydrogen in the reaction, the reactor may be operated horizontally and may be open at the top; in this case, the catalyst is fixed by a mesh. The hydrogen can also be driven out by gases such as nitrogen, argon, but also hydrogen.

### EXAMPLES

The following examples are illustrative and are intended to be non-limiting.

The Pt—S/La—Al₂O₃ catalyst of the present disclosure consists of Pt metal (0.01-2.00 wt. %) or metal oxide dispersed on lanthanum oxide doped Al₂O₃ (0.01-8.00 wt. % La₂O₃) that has been additionally treated with sulfur compounds to achieve an S content in the finished product of 0.01-2.00 wt. %.

### Example 1

### Catalyst Preparation

A Pt—S/La—Al₂O₃ catalyst was prepared by first extruding a base material consisting of lanthanum-doped Al₂O₃ (La—Al₂O₃), either with or without the addition of sulfate precursors H₂SO₄ or (NH₄)₂SO₄, and subsequently impregnating the solid extrudates with aqueous Pt(NH₃)₄(NO₃)₂ buffered by NH₄OH and HNO₃ (to achieve pH 9.2-9.4) in order to reach the desired Pt loading. The metal-extruded sample is finally dried (at ˜150° C., 1-8 h) and then calcined (in air at 385-455° C., 20-60 min) to create the finished product. If sulfur was not added to the base material during the extrusion phase of the preparation, then sulfur may be added to the base material during the metal impregnation step by co-impregnating (NH₄)₂SO₄ together with Pt(NH₃)₄(NO₃)₂, or alternatively, by carrying out a two-step impregnation/calcination procedure whereby S and then Pt precursors are sequentially added to the extruded base.

The following comparative catalysts were prepared analogously to Pt—S/La—Al₂O₃ with 0.5 wt. % Pt loading: a sulfur-free catalyst (Pt/La—Al₂O), a sulfur- and lanthanum-free catalyst containing Al₂O₃ doped by silica (SiO₂; 5.0 wt. %) rather than La₂O₃, and a sulfur- and lanthanum-free catalyst containing Al₂O₃ doped by cerium oxide (CeO₂; 4.4 wt. %).

The chemical and physical properties of the catalysts are reported in Table 1.

TABLE 1
Catalyst Properties

| | | | | Pt—S/ |
|---|---|---|---|---|
| | Catalyst | Pt/Al₂O₃ | Pt/Al₂O₃ | Al₂O₃ |
| | Pt/ | (La- | (Ce- | (La- |
| | SiO₂—Al₂O₃ | doped) | doped) | doped) |
| Base Dopant | 5.0 | 5.0 | 4.4 | 5.0 |
| [wt. % oxide] | | | | |
| Pt [wt. %] | 0.5 | 0.5 | 0.5 | 0.5 |
| S Theor. [wt. %] | 0 | 0 | 0 | 0.5 |
| S Actual [wt. %] | 0 | 0 | 0 | 0.39 |
| Surface area | 281 | 270 | 247 | 270 |
| [m²/g] | | | | |
| Density [g/cm³] | 0.59 | 0.98 | 0.94 | 0.98 |
| Dispersion | 124 | 129.8 | 115.3 | 24.6 |
| (total H₂) | | | | |
| [%] | | | | |
| Dispersion | 75.1 | 80.9 | 77.7 | 9.4 |
| (strong H₂) | | | | |
| [%] | | | | |
| mg Pt/cm³ | 3.0 | 4.9 | 4.9 | 4.9 |
| mg Pd/cm³ | 0 | 0 | 0 | 0 |
| mg Ni/cm³ | 0 | 0 | 0 | 0 |
| mg metal/cm³ | 3.0 | 4.9 | 4.9 | 4.9 |
| μmol_{S-metal}/cm³ | 11.5 | 20.2 | 18.8 | 2.3 |

Metal dispersion was assessed using hydrogen chemisorption measurements. The metal dispersion estimates the ratio of the number of active metal atoms available for reaction to the total number of metal atoms in the catalyst material. The metal percent dispersion is the ratio of the available quantity to total quantity of active molecules times 100%. A metal dispersion over 100% (total H₂) indicates weak or physical adsorption. Weak adsorption is termed reversible adsorption, whereas strong chemisorption is termed irreversible adsorption. The total chemisorption is the combination of weak and strong adsorption.

The metal dispersion was determined using the following procedure: the catalyst was first calcined under He and reduced under hydrogen. After the catalyst was evacuated for 1 hour, the catalyst was held under vacuum until the total H2 chemisorption was measured at 35° C. for the following pressures: 20, 50, 80, 110, 140, 170, 200, 240, 280, 320 and 360 torr. After the total chemisorption was measured, the catalyst was evacuated for 10 minutes and the isothermal H₂ chemisorption was repeated to determine the weak and strong H₂ chemisorption components. The metal dispersion was calculated by the strongly adsorbed hydrogen per Pt atom.

### Example 2

### Catalytic Testing

The activity of the catalysts was evaluated as follows. Bench-scale unit (BSU) dehydrogenation of perhydro-dibenzyltoluene (H₁₈-DBT) was performed using conditions listed in Table 2.

TABLE 2
Dehydrogenation Conditions

| Test Parameter | Parameter Value | |
|---|---|---|
| Total Pressure | 50 | psig |
| LHSV | 0.1 | h⁻¹ |
| H₂ Supply Rate | 6000 SCF H₂/BBL | |
| Pretreatment | H₂, 600° F., 8 h | |
| Catalyst Charge | 30 | cm³ |

| | |
|---|---|
| Reaction C.A.T Targets | 450° F.-650° F. |

The properties of the single component perhydro-dibenzyltoluene feed and the desired product (dibenzyltoluene) are listed in Table 3.

TABLE 3
Properties of Perhydro-Dibenzyltoluene
(H₁₈-DBT) and Dibenzyltoluene (H₀-DBT)

| | Perhydro- | |
|---|---|---|
| | Dibenzyltoluene | Dibenzyltoluene |
| Boiling Point [° F.] | 700 | 734 |
| Density [g/cm³] | 0.91 | 1.04 |
| Mol Wt. [g/mol] | 290 | 272 |
| H [wt. %] | 13.1 | 7.35 |

The degree of dehydrogenation (DoDH), used to quantify the extent to which perhydro-dibenzyltoluene has been converted into its fully unsaturated analogue, dibenzyltoluene, is defined here as:

$$DoDH ( \% ) = 100 * \frac{( 13.103 - [ H ] )}{( 13.103 - 7.353 )}$$

where [H] is the hydrogen content (in wt. %) of the product liquid generated by the dehydrogenation catalyst.

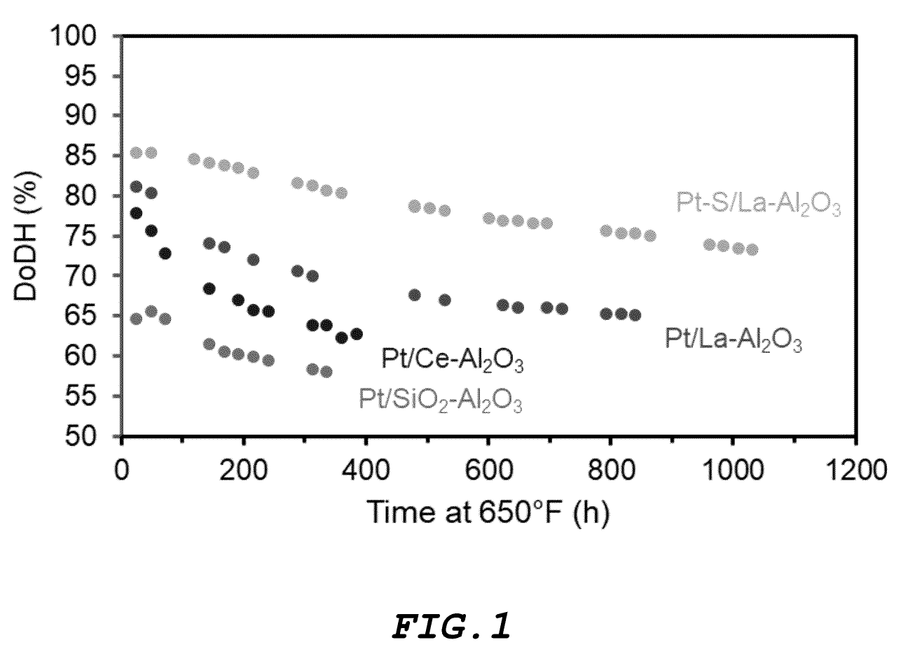

The dehydrogenation performance results at 650° F. are summarized in FIG. 1 and Table 4.

A review of FIG. 1 reveals that the Pt—S/La—Al₂O₃ catalyst gave higher initial DoDH of perhydro-dibenzyltoluene (85.4%) than Pt/La—Al₂O₃ (80.4%), Pt/Ce—Al₂O₃ (77.8%), and Pt/SiO₂—Al₂O₃ (65.6%) catalysts at 650° F.

TABLE 4
Catalyst Activity Loss for Dehydrogenation of
Perhydro-Dibenzyltoluene (H₁₈-DBT) at 650° F.

Activity Loss at 300 h Time-on-Stream

| Catalyst | Fractional | Absolute |
|---|---|---|
| Pt—S/La—Al₂O₃ | 4.6% | −3.9% |
| Pt/La—Al₂O₃ | 12.6% | −10.1% |
| Pt/Ce—Al₂O₃ | 17.8% | −13.8% |
| Pt/SiO₂—Al₂O₃ | 10.8% | −7.1% |

A review of Table 4 reveals that the Pt—S/La—Al₂O₃ catalyst experienced a loss in DoDH conversion (−3.9%) that is less severe than that for the Pt/La—Al₂O₃ (−10.1%), Pt/Ce—Al₂O₃ (−13.8%), and Pt/SiO₂—Al₂O₃ (−7.1%) catalysts in absolute terms. In proportional terms, the Pt—S/La—Al₂O₃ catalyst retains a larger proportion of its initial activity (95.4%) than the Pt/La—Al₂O₃ (87.4%), Pt/Ce—Al₂O₃ (82.2%), and Pt/SiO₂—Al₂O₃ (89.2%) catalysts.

In summary, the results in FIG. 1 and Table 4 indicate that doping of both the alumina support by lanthanum and the Pt metal hydrogenation/dehydrogenation function by sulfur contribute to the improved reaction characteristics of Pt—S/La—Al₂O₃ relative to the alternative catalysts. The doubly doped Pt—S/La—Al₂O₃ catalyst is more active and resistant to deactivation than Pt/La—Al₂O₃, Pt/Ce—Al₂O₃, and Pt/SiO₂—Al₂O₃ variants.

1. A supported noble metal catalyst comprising:
(a) a carrier, wherein the carrier is a shaped structure defined by a shape and comprises an inorganic oxide and a non-noble metal dopant, wherein the non-noble metal dopant is present in an amount of from 0.01 to 10 wt. % of catalyst, measured as oxide.
(b) a noble metal component, wherein a content of the noble metal component is from 0.01 to 2 wt. %, measured as elemental metal; and
(c) a sulfur-containing component, wherein a content of the sulfur-containing component is from 0.01 to 2 wt. %, measured as elemental sulfur.

2. The catalyst of claim 1, wherein the shape is selected from the group consisting of spheres, cylindrical pellets, and extrudates.

3. The catalyst of claim 1, wherein the inorganic oxide is selected from the group consisting of alumina, silicon dioxide, silica-alumina, titanium dioxide, zirconium dioxide, silicon carbide, and any combination thereof.

4. The catalyst of claim 1, wherein the inorganic oxide is alumina.

5. The catalyst of claim 4, wherein the alumina is selected from the group consisting of alpha-alumina, beta-alumina, gamma-alumina, delta-alumina, theta-alumina, and any combination thereof.

6. The catalyst of claim 1, wherein the non-noble metal dopant comprises titanium (Ti), zirconium (Zr), chromium (Cr), manganese (Mn), iron (Fe), cobalt (Co), nickel (Ni), copper (Cu), zinc (Zn), tin (Sn), scandium (Sc), yttrium (Y), lanthanum (La), praseodymium (Pr), neodymium (Nd), samarium (Sm), gadolinium (Gd), ytterbium (Yb), or metal oxides thereof.

7. The catalyst of claim 1, wherein the noble metal is selected from the group consisting of ruthenium, iridium, palladium, platinum, and any combination thereof.

8. The catalyst of claim 1, wherein a molar ratio of noble metal to sulfur is in a range of from 1:1 to 1:5, and wherein the sulfur-containing component is calculated as elemental sulfur.

9. The catalyst of claim 1, wherein a source of the sulfur-containing component is selected from the group consisting of sulfuric acid, ammonium sulfate, and a combination thereof.

10. A process for the hydrogenation of an aromatic compound comprising
(a) preparing a mixture of an aromatic compound and a supported metal catalyst according to claim 1; and
(b) contacting the mixture obtained in (a) with hydrogen at a temperature comprised in the range of from 100° C. to 350° C.

11. The process of claim 1, wherein the aromatic compound is selected from the group consisting of benzene, toluene, benzyltoluene, dibenzyltoluene, xylene, naphthalene, fluorene, N-alkylcarbazoles, light cycle oils, and any combination thereof.

12. The process of claim 10, wherein the catalyst is used in the amount of 0.01 part by weight to 10 parts by weight based on 100 parts by weight of the aromatic compound or the unsaturated compound.

13. The process of claim 10, wherein the hydrogenation is carried out at a pressure of from 0.1 MPa to 10 MPa.

14. A process for producing hydrogen, the process comprising:
contacting a feed comprising a perhydrogenated or partly hydrogenated cyclic hydrocarbon under dehydrogenation conditions with a supported noble metal catalyst according to claim 1.

15. The process of claim 14, wherein the perhydrogenated or partly hydrogenated cyclic hydrocarbon is selected from the group consisting of cyclohexane, methylcyclohexane, decalin, perhydrogenated or partly hydrogenated benzyltoluene, perhydrogenated or partly hydrogenated dibenzyltoluene, perhydrogenated or partly hydrogenated N-alkylcarbazoles, perhydrogenated or partly hydrogenated light cycle oils and any combination thereof.

16. The process of claim 14, wherein the dehydrogenation conditions comprise a temperature of from 200° C. to 400° C., a pressure of from 50 kPa to 500 kPa, and a liquid hourly space velocity of from 0.1 h⁻¹ to 3 h⁻¹.
